(19) United States
(12) Patent Application Publication
(10) Pub. No.: US 2023/0049153 A1
(43) Pub. Date: Feb. 16, 2023

(54) QUANTITATIVE KIT FOR MYXOVIRUS RESISTANCE PROTEIN 1

(71) Applicant: Beijing Strong Biotechnologies, Inc., Beijing (CN)

(72) Inventors: Jun Gong, Beijing (CN); Jinxiang Qi, Beijing (CN); Lanping Xiao, Beijing (CN); Guili Wang, Beijing (CN); Xi LlU, Beijing (CN)

(21) Appl. No.: 17/790,479

(22) PCT Filed: Nov. 4, 2020
(86) PCT No.: PCT/CN2020/126382

§ 371 Date: Jun. 30, 2022

(30) Foreign Application Priority Data
Jan. 20, 2020 (CN) 202010063962.7

(51) Int. Cl. G01N 33/543; G01N 33/68; G01N 33/577

(52) U.S. Cl. CPC G01N 33/54313; G01N 33/68; G01N 33/577

(57) ABSTRACT

The present application relates to a quantitative kit for myxovirus resistance protein 1. Specifically, a kit comprising latex particles coated with a myxovirus resistance protein 1 antibody is disclosed. Myxovirus resistance protein 1 in human serum and plasma samples and latex particles cross-linked with a myxovirus resistance protein 1 antibody are specifically binded to form a complex, which leads to an increase in absorbance. By detecting changes in immunoturbidity, a higher sensitivity and a wider detection range are reached.

### FIELD OF THE INVENTION

The present application belongs to the field of in vitro medical immuno-diagnosis, and provides a kit for measuring the content of Myxovirus Resistance Protein 1 in a sample by using latex-enhanced turbidimetric immunoassay.

### BACKGROUND OF THE INVENTION

Myxovirus Resistance Protein 1 is a natural protein, widely present in tissues and cells, consisting of 662 amino acids with a molecular weight of about 78 kD (Sun Shaomei et al., Research and Application of Mx1 Antiviral Protein, Chinese Journal of Zoological Diseases, 2011, 27 (4):351-354).

Once the body is infected by a virus, IFN is significantly increased, and interferon-α (IFN-α) binds to the receptor proteins IFNAR1 and IFNAR2 to initiate the expression of gene MX1 to form MX1. The level of MX1 protein in vivo is helpful for evaluating the efficacy of IFN. Mx1 protein is closely related to virus infection, and shows very sensitive response to virus. Even a very small amount of virus can induce cells to express Mx1 protein, so it can be used for diagnosis of early virus infection, and it can be used clinically for identification diagnosis of virus and bacterial infection (Mao Guoqiang et al., Foreign Medical Virology Volume 2005, Vol 12: 107-109). Therefore, Myxovirus Resistance Protein was identified for the first time as a potential biomarker to distinguish bacterial infection from viral infection at the 68th World Health Congress in 2015 (Clin Chem. 2019 June; 65 (6):739-750. doi:10.1373/clinchem.2018.292391. Epub 2018 Dec. 28).

CN106442984A discloses the use of an antibody for detecting the expression level of MX1 in combination with an antibody for detecting the expression level of CRP for distinguishing bacterial (or mixed) infection from viral infection.

At present, Enzyme-Linked Iimmunosorbent Assay (ELISA) is the mainly clinical diagnosis technology for MX1. However, the popularization of MX1 in clinical testing is severely restricted due to the cumbersome operation, long time-consuming, susceptible to external factors and personnel operations, and low degree of automation.

### SUMMARY OF THE INVENTION

According to some embodiments of the present application, there is provided a kit for measuring the content of Myxovirus Resistance Protein 1 in a human sample.

In a specific embodiment, there is provided a kit for measuring the content of Myxovirus Resistance Protein 1 in a human sample, comprising:
a first reagent,
a second reagent, and
optionally, a calibrator and/or quality control;
wherein:
the first reagent comprises:
10 mM to 200 mM buffer,
0.1% to 15% w/v stabilizer,
1% to 6% w/v coagulant, and
0.02% to 0.1% w/v preservative;
the second reagent comprises:
0.05% to 0.5% w/v latex particles coated with an antibody against Myxovirus Resistance Protein 1,
10 mM to 200 mM buffer,
0.1% to 15% w/v stabilizer, and
0.02% to 0.1% w/v preservative;
the calibrator comprises:
Myxovirus Resistance Protein 1 with known concentration(s),
10 mM to 200 mM buffer,
0.1% to 15% w/v stabilizer, and
0.02% to 0.1% w/v preservative.

In some embodiments, the antibody against Myxovirus Resistance Protein 1 is derived from: murine, monkey, caprinae, leporinae, bovine, swine, poultry, camelid, or recombinant antibody.

In some embodiments, the antibody against Myxovirus Resistance Protein 1 is coated on the surface of the latex particles, preferably the antibody against Myxovirus Resistance Protein 1 is covalently coupled to the surface of the latex particles.

In some embodiments, the latex particles have an average particle size of 50 nm to 350 nm (50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350 nm).

In some embodiments, the surface of the latex particles has one or a combination of chemical groups selected from the group consisting of: carboxyl group, amino group, hydroxyl group, hydrazide group and chloromethyl group.

In some embodiments, the buffer is selected from one or a combination of the following: tromethamine buffer, phosphate buffer, Tris-HCl buffer, citric acid-sodium citrate buffer, barbiturate buffer, glycine buffer, borate buffer and trihydroxymethyl methane buffer.

In some embodiments, the stabilizer is selected from one or a combination of the following: 0.1% to 5% w/v bovine serum albumin, 5% to 10% w/v trehalose, 10% to 20% w/v glycerol, 5% to 10% w/v sucrose, 5% to 10% w/v mannitol, 5% to 15% w/v glycine and 5% to 15% w/v arginine.

In some embodiments, the preservative is selected from one or a combination of the following: sodium azide, thimerosal, phenol, ethyl mercury and sodium thiosulfate.

In some embodiments, the coagulant is selected from one or a combination of the following: PEG-4000, PEG-6000, PEG-8000 and glucan.

In the present application, the antibody also includes the category of antigen-binding fragments.

In some embodiments, the detection reagents of the present application are useful for the quantitative or qualitative determination of the amount of, the presence or absence of, and the expression level of Myxovirus Resistance Protein 1 in human samples (serum, plasma, urine, cerebrospinal fluid, secretion, tissue fluid, saliva, biopsy sample, tear, excreta, sweat, cystic fluid).

### DETAILED DESCRIPTION OF THE INVENTION

### Example 1. Preparation of the Kit

1. Preparation of the First Reagent:

0.9% sodium chloride, 5% PEG-6000, 2% BSA, and 0.1% sodium azide were added to 50 mM Tris-HCl buffer pH 7.2, and mixed well to obtain the first reagent.

2. Preparation of Second Reagent

0.5 mL of polystyrene latex solution (at the concentration of 10%, purchased from JSR), with an average particle size of 335 nm and modified with carboxyl group on the surface, was added into 4.5 mL of 0.05 M MES buffer pH 6.0, and then 5 mg EDAC was added to react at 37° C. for 1 hour;

The unreacted EDAC was removed by centrifuging at 15,000 rpm, and 5 ml of coupling buffer (glycine buffer, pH 8.0) was added for resuspension;

Then 0.5 mg of the antibody against Myxovirus Resistance Protein 1 (commercially available antibody) was immediately added to the above latex solution, to react at 37° C. for 1 hour;

The free antibody was removed by centrifuging at 15,000 rpm, and finally 5 mL of 2% BSA blocking solution was added to resuspend the pellet;

The supernatant was removed by centrifuging at 15,000 rpm, the pellet was washed three times with 20 mL of 50 mM glycine buffer pH 8.0 (comprising 0.9% sodium chloride, 2% BSA, 0.1% Tween 20, 0.1% sodium azide), and then was dispersed in 20 mL of the same glycine buffer to obtain a milky white latex suspension, resulting in the second reagent (the concentration of the latex particle is 0.25%).

3. Calibrator

Myxovirus Resistance Protein 1 was added to 50 mM Tris-HCl buffer pH 7.2 at concentrations of 0, 25, 50, 100, 200, 400 ng/L, and then 2% BSA, 150 mM sodium chloride and 0.1% sodium azide were added, mixed well to obtain calibrator(s) for Myxovirus Resistance Protein 1.

The above reagents were assembled into a kit.

### Example 2. Preparation of the Control Kit

Similarly to Example 1, except that the antibody epitope, the concentration of each component, or the particle size of the latex particles were changed.

### Example 3. Performance Test of MX1 Kit

1. Test Method

Hitachi 7180 biochemical analyzer was used as an example: the measurement wavelength was 570 nm. Firstly, 180 μl of the first reagent and 3.0 μl of the calibrator were added, to react at 37° C. for 5 min, and then 60 μl of the second reagent was added.

The absorbance values A1 and A2 at 353 seconds and 600 seconds of the reaction were measured, and the absorbance difference ΔA=A2−A1 was calculated. The measurement for each tube was repeated twice, and the concentration-absorbance difference calibration curve was plotted, with the absorbance difference ΔA measured twice for each calibration tube as the vertical coordinate, and the corresponding concentration as the abscissa. Similarly, the absorbance difference was measured for the sample to be tested, and the amount of MX1 in the sample to be tested can be calculated by fitting against the calibration curve.

2. Repeatability

MX1 was diluted with normal saline to get five concentration gradient levels of 30, 60, 120, 180, and 360 ng/ml, respectively. After calibration on Hitachi, five concentration gradient levels of MX1 samples were detected, each of which was detected for 21 times, and the mean and coefficient of variation were calculated respectively.

TABLE 1
Detection repeatability of the Kit of the Present Application

| Test | 30 ng/ml | 60 ng/ml | 120 ng/ml | 180 ng/ml | 360 ng/ml |
|---|---|---|---|---|---|
| 1 | 30.32 | 58.17 | 118.12 | 176.45 | 359.01 |
| 2 | 28.57 | 59.41 | 124.45 | 180.24 | 349.24 |
| 3 | 30.14 | 60.42 | 121.57 | 176.17 | 351.21 |
| 4 | 30.02 | 61.24 | 117.94 | 179.18 | 348.57 |
| 5 | 29.17 | 59.57 | 121.78 | 183.39 | 358.69 |
| 6 | 28.78 | 61.34 | 122.25 | 176.14 | 361.14 |
| 7 | 29.31 | 59.57 | 121.67 | 182.78 | 351.24 |
| 8 | 29.14 | 60.54 | 122.45 | 179.47 | 353.47 |
| 9 | 30.35 | 61.15 | 124.69 | 176.74 | 362.26 |
| 10 | 29.17 | 60.68 | 117.52 | 176.84 | 364.47 |
| 11 | 30.08 | 58.87 | 121.45 | 183.67 | 346.24 |
| 12 | 29.95 | 62.54 | 124.46 | 185.24 | 347.17 |
| 13 | 30.47 | 59.87 | 120.39 | 178.87 | 359.24 |
| 14 | 30.87 | 61.7 | 124.59 | 177.71 | 355.51 |
| 15 | 29.04 | 60.14 | 118.01 | 183.54 | 367.31 |
| 16 | 28.65 | 61.58 | 120.48 | 183.83 | 352.14 |
| 17 | 29.74 | 58.24 | 121.79 | 178.79 | 347.17 |
| 18 | 30.98 | 58.96 | 118.12 | 181.14 | 364.28 |
| 19 | 30.17 | 60.79 | 124.18 | 183.24 | 349.39 |
| 20 | 30.24 | 58.92 | 120.49 | 178.18 | 355.27 |
| 21 | 29.41 | 57.92 | 120.74 | 179.59 | 346.67 |
| mean | 29.74 | 60.08 | 121.29 | 180.06 | 354.75 |
| SD | 0.71 | 1.28 | 2.37 | 2.95 | 6.63 |
| CV | 2.40% | 2.14% | 1.96% | 1.64% | 1.87% |

3. Linearity

A high concentration sample with a concentration of about 400 ng/ml was prepared by adding MX1, 10 arithmetical dilutions were made with normal saline, to prepare 11 levels of linear samples; and the concentration of each level was measured for 3 times for each sample. The deviation between the mean and the theoretical value was calculated.

TABLE 2
Detection linearity of the Kit of the Present Application

| | Theoretical | Detection | |
|---|---|---|---|
| Dilution | concentration | concentration | Deviation |
| ratio | (ng/ml) | (ng/ml) | (%) |
| 0/10 | 0 | 0.1 | |
| 1/10 | 39.80 | 0.1 | |
| 2/10 | 79.60 | 38.42 | 3.46 |
| 3/10 | 119.40 | 81.30 | −2.14 |
| 4/10 | 159.20 | 116.83 | 2.15 |
| 5/10 | 199.00 | 164.25 | −3.17 |
| 6/10 | 238.80 | 196.53 | 1.24 |
| 7/10 | 278.60 | 241.28 | −1.04 |
| 8/10 | 318.40 | 272.53 | 2.18 |
| 9/10 | 358.20 | 323.43 | −1.58 |
| 10/10 | 398.00 | 345.77 | 3.47 |

It can be seen from Table 1 and Table 2 that the detection kit of the present application performs well in detection repeatability and linearity, and can provide a good choice for the clinical detection of MX1.

1. A kit for quantitatively determining Myxovirus Resistance Protein 1, the kit comprising latex particles coated with an antibody against Myxovirus Resistance Protein 1.

2. The kit for quantitatively determining Myxovirus Resistance Protein 1 according to claim 1, comprising:
a first reagent,
a second reagent, and
optionally, a calibrator and/or quality control;
wherein:
the first reagent comprises:
10 mM to 200 mM buffer,
0.1% to 15% w/v stabilizer,
1% to 6% w/v coagulant, and
0.02% to 0.1% w/v preservative;
the second reagent comprises:
0.05% to 0.5% w/v latex particles coated with an antibody against Myxovirus Resistance Protein 1,
10 mM to 200 mM buffer,
0.1% to 15% w/v stabilizer, and
0.02% to 0.1% w/v preservative;
the calibrator comprises:
Myxovirus Resistance Protein 1 with known concentration(s),
10 mM to 200 mM buffer,
0.1% to 15% w/v stabilizer, and
0.02% to 0.1% w/v preservative;
the antibody against Myxovirus Resistance Protein 1 is derived from: murine, monkey, caprinae, leporinae, bovine, swine, poultry, camelid, or recombinant antibody;
the antibody against Myxovirus Resistance Protein 1 is coated on the surface of the latex particles, preferably the antibody against Myxovirus Resistance Protein 1 is covalently coupled to the surface of the latex particles;
the average particle size of the latex particles is from 50 nm to 350 nm;
the surface of the latex particles has one or a combination of chemical groups selected from the group consisting of: carboxyl group, amino group, hydroxyl group, hydrazide group and chloromethyl group.

3. The kit for quantitatively determining Myxovirus Resistance Protein 1 according to claim 2, comprising:
a first reagent,
a second reagent, and
a calibrator;
wherein:
the first reagent comprises:
0.9% w/v sodium chloride,
5% w/v PEG-6000,
2% w/v BSA,
0.1% w/v sodium azide,
50 mM Tris-HCl buffer pH 7.2;
the second reagent comprises:
0.25% w/v polystyrene latex particles coated with an antibody against Myxovirus Resistance Protein 1,
50 mM glycine buffer pH 8.0,
0.9% w/v sodium chloride,
2% w/v BSA,
0.1% w/v Tween 20,
0.1% w/v sodium azide;
the calibrator comprises:
0, 25, 50, 100, 200, 400 ng/ml Myxovirus Resistance Protein 1,
1% w/v BSA,
150 mM sodium chloride,
0.1% w/v sodium azide,
50 mM Tris-HCl buffer pH 7.20;
wherein, the surface of the latex particles is modified with carboxyl group;
the average particle size of the latex particles is 350 nm.

4. The kit for quantitatively determining Myxovirus Resistance Protein 1 according to claim 2, wherein:
the buffer is selected from one or a combination of the following: tromethamine buffer, phosphate buffer, Tris-HCl buffer, citric acid-sodium citrate buffer, barbiturate buffer, glycine buffer, borate buffer and trihydroxymethyl methane buffer.

5. The kit for quantitatively determining Myxovirus Resistance Protein 1 according to claim 2, wherein:
the stabilizer is selected from one or a combination of the following: 0.1% to 5% w/v bovine serum albumin, 5% to 10% w/v trehalose, 10% to 20% w/v glycerol, 5% to 10% w/v sucrose, 5% to 10% w/v mannitol, 5% to 15% w/v glycine and 5% to 15% w/v arginine.

6. The kit for quantitatively determining Myxovirus Resistance Protein 1 according to claim 2, wherein:
the preservative is selected from one or a combination of the following: sodium azide, thimerosal, phenol, ethyl mercury and sodium thiosulfate.

7. The kit for quantitatively determining Myxovirus Resistance Protein 1 according to claim 2, wherein:
the coagulant is selected from one or a combination of the following: PEG-4000, PEG-6000, PEG-8000 and glucan.
